(19) United States
(12) Patent Application Publication
(10) Pub. No.: US 2021/0218060 A1
(43) Pub. Date: Jul. 15, 2021

(54) ELECTROLYTE COMPOSITION AND USE THEREOF IN LITHIUM-ION BATTERIES

(71) Applicants: HYDRO-QUÉBEC, Montréal (CA); ARKEMA FRANCE, Colombes (FR)

(72) Inventors: Sabrina PAILLET, Lescar (FR); Gregory SCHMIDT, St Andéol Le Château (FR); Ian CAYREFOURCQ, St Nazaire Les Eymes (FR); Julie HAMEL-PÂQUET, Montréal (CA); Ali DARWICHE, Longueuil (CA); Gabriel GIRARD, Longueuil (CA); Joël FRÉCHETTE, Boucherville (CA); Sébastien LADOUCEUR, Varennes (CA); Abdelbast GUERFI, Brossard (CA); Karim ZAGHIB, Longueuil (CA)

(73) Assignees: HYDRO-QUÉBEC, Montréal, QC (CA); ARKEMA FRANCE, Colombes (FR)

(21) Appl. No.: 16/492,227

(22) PCT Filed: Mar. 9, 2018
(86) PCT No.: PCT/IB2018/051573

§ 371 Date: Sep. 9, 2019

(30) Foreign Application Priority Data
Mar. 10, 2017 (CA) 2960489
Mar. 10, 2017 (FR) 17/51971

(51) Int. Cl. H01M 10/0568; C07D 233/90; H01M 10/0525; H01M 10/0569

(52) U.S. Cl. CPC H01M 10/0568; C07D 233/90; H01M 2300/0028; H01M 10/0569; H01M 10/0525

(57) ABSTRACT

Electrolyte compositions comprising lithium hexafluorophosphate, lithium 4,5-dicyano-2-(trifluoromethyl)imidazolate, a solvent, and at least one electrolytic additive, are herein described. The present application also describes the use if these electrolyte compositions in batteries, e.g. within a temperature range higher than or equal to 25° C.

### RELATED APPLICATIONS

The present application claims priority under applicable law to Canadian patent application No. 2 960 489 filed on Mar. 10, 2017 and to French patent application No. 17 51971 also filed on Mar. 10, 2017, the content of which is incorporated herein by reference in its entirety for all purposes.

### TECHNICAL FIELD

The present application relates to the field of batteries, more particularly to the field of electrolyte compositions comprising lithium ions.

### TECHNICAL BACKGROUND

A lithium-ion battery comprises at least a negative electrode (anode), a positive electrode (cathode), a separator and an electrolyte. The electrolyte generally consists of a lithium salt dissolved in a solvent which is usually a mixture of organic carbonates, in order to have a good compromise between viscosity and dielectric constant. Additives can then be added to improve stability of the electrolyte salts.

Among the most used salts is LiPF₆ (lithium hexafluorophosphate), which possesses many of the required qualities but has the disadvantage of degrading to form hydrofluoric acid (HF) by reacting with water. The formed HF can cause a dissolution of the cathode material. The reaction of LiPF₆ with residual water thus affects the longevity of the battery and can cause safety problems, especially when lithium-ion batteries are used in private vehicles.

Other salts, such as LiTFSI (lithium bis(trifluoromethanesulfonyl)imide) and LiFSI (lithium bis(fluorosulfonyl)imide), have thus been developed. These salts show little or no spontaneous decomposition and are more stable to hydrolysis than LiPF₆. Nevertheless, LiTFSI has the disadvantage of being corrosive for current collectors, particularly those in aluminum.

In the field of batteries, there is an ongoing need for developing electrolyte compositions for improving battery performance, such as its durability, its cycling stability, and/or the reduction of its irreversible capacity.

### SUMMARY

The present application relates to an electrolyte composition comprising lithium hexafluorophosphate, lithium 4,5-dicyano-2-(trifluoromethyl)imidazolate, at least one solvent, and at least one electrolytic additive, said composition comprising:
a total concentration of lithium hexafluorophosphate and lithium 4,5-dicyano-2-(trifluoromethyl)imidazolate of less than or equal to 1 mol/L relative to the total volume of composition, and
a concentration of lithium 4,5-dicyano-2-(trifluoromethyl)imidazolate of less than or equal to 0.3 mol/L relative to the total volume of composition.

According to one embodiment, the content of lithium 4,5-dicyano-2-(trifluoromethyl)imidazolate is less than or equal to 0.2 mol/L, in particular less than or equal to 0.1 mol/L, preferably less than or equal to 0.08 mol/L, more preferably less than or equal to 0.05 mol/L, relative to the total volume of composition.

According to another embodiment, the composition solvent is selected from the group consisting of ethers, carbonic acid esters, cyclic carbonate esters, aliphatic carboxylic acid esters, aromatic carboxylic acid esters, phosphoric acid esters, sulfite esters, nitriles, amides, alcohols, sulfoxides, sulfolane, nitromethane, 1,3-dimethyl-2-imidazolidinone, 1,3-dimethyl-3,4,5,6-tetrahydro-2(1,H)-pyrimidinone, 3-methyl-2-oxazolidinone, and mixtures thereof. For example, the solvent is selected from the group consisting of dimethyl carbonate, ethyl methyl carbonate, diethyl carbonate, diphenyl carbonate, methyl phenyl carbonate, ethylene carbonate, propylene carbonate, butylene carbonate, vinylene carbonate, methyl formate, methyl acetate, methyl propionate, ethyl acetate, butyl acetate, and mixtures thereof. The solvent may also be selected from ethylene carbonate, diethyl carbonate, and mixtures thereof.

In another embodiment, the electrolytic additive is selected from the group consisting of fluoroethylene carbonate, vinylene carbonate, 4-vinyl-1,3-dioxolan-2-one, allyl ethyl carbonate, vinyl acetate, divinyl adipate, acrylonitrile, 2-vinylpyridine, maleic anhydride, methyl cinnamate, phosphonates, vinyl containing silane compounds, 2-cyanofurane and mixtures thereof, the electrolytic additive preferably being fluoroethylene carbonate. For instance, the content of electrolytic additive is comprised between 0.1% and 9%, preferably between 0.5% and 4% by weight relative to the total combined weight of solvent(s) and additive.

In an embodiment, the concentration of lithium hexafluorophosphate in the electrolyte composition is greater than or equal to 0.80 mol/L and less than 1 mol/L, preferably comprised between 0.80 and less than 1 mol/L, particularly between 0.90 and 0.99 mol/L, and for example comprised between 0.95 mol/L and 0.99 mol/L. For instance, the lithium hexafluorophosphate concentration is of about 0.95 mol/L, and the lithium 4,5-dicyano-2-(trifluoromethyl)imidazolate concentration is of about 0.05 mol/L, relative to the total volume of composition.

The present application also relates to the use of a composition as defined herein, in a Li-ion battery, particularly in a temperature range above or equal to 25° C., preferably comprised between 25° C. and 65° C., advantageously between 40° C. and 60° C. For example, use is made in mobile devices, for instance mobile phones, cameras, tablets or laptops, in electric vehicles, or in the storage of renewable energy. Another embodiment comprises the use of a composition as defined in the present application for improving life duration of a Li-ion battery; and/or improving cycling stability of a Li-ion battery; and/or reducing irreversible capacity of a Li-ion battery; particularly in a temperature range above or equal to 25° C., preferably comprised between 25° C. and 65° C., advantageously between 40° C. and 60° C.

Another aspect of the present application relates to an electrochemical cell comprising a negative electrode, a positive electrode, and an electrolyte composition as defined herein, interposed between the negative electrode and the positive electrode.

In one embodiment, the negative electrode of the electrochemical cell comprises graphite, carbon fibers, carbon black, lithium, or a mixture thereof, the negative electrode preferably comprising graphite.

In another embodiment, the electrochemical cell positive electrode comprises LiCoO₂, LiFePO₄ (LFP), LiMnₓCo_{y}Ni_{z}O₂ (NMC, where x+y+z=1), LiFePO₄F, LiFeSO₄F, LiNiCoAlO₂ or a mixture thereof, the positive electrode preferably comprising LiFePO₄ or LiMnₓCo_{y}Ni_{z}O₂ (where x+y+z=1).

For example, the electrochemical cell as described herein may have a capacity retention greater than or equal to 80% after at least 500 charge/discharge cycles relative to the first cycle, for a charge comprised between a voltage V_{low} comprised between 2.0 and 3.0 volts versus Li⁺/Li⁰, and a voltage V_{high} comprised between 3.8 and 4.2 volts versus Li⁺/Li⁰, at a temperature of 25° C., and at a charge and discharge C rate. For instance, the voltage V_{low} is equal to 2.8 volts and the voltage V_{high} is equal to 4.2 volts, the positive electrode preferably comprising LiCoO₂, LiMnₓCo_{y}Ni_{z}O₂ (with x+y+z=1), LiFePO₄F, LiFeSO₄F, LiNiCoAlO₂ and mixtures thereof.

According to another example, the electrochemical cell has a capacity retention greater than or equal to 80% after at least 500 charge/discharge cycles compared to the first cycle, for a charge comprised between a voltage V_{low} between 2.0 and 3.0 volts versus Li⁺/Li⁰, and a voltage V_{high} between 3.8 and 4.2 volts versus Li⁺/Li⁰, at a temperature of 25° C., and at a charge and discharge C rate, charging being optionally followed by the application of a constant voltage of 4V during 30 minutes, the positive electrode preferably comprising LiFePO₄. According to an example, the voltage V_{low} is equal to 2 volts and the voltage V_{high} is equal to 4 volts. According to an embodiment, charging is followed by the application of a constant voltage of 4V for 30 minutes. According to another embodiment, charging is not followed by the application of a constant voltage and the capacity retention is greater than or equal to 80% relative to the first cycle after at least 800 charge/discharge cycles.

According to another aspect, this application also relates to a battery comprising at least one electrochemical cell as described in the present application.

Another aspect relates to the use of lithium 4,5-dicyano-2-(trifluoromethyl)imidazolate in an electrolyte composition comprising lithium hexafluorophosphate and at least one electrolytic additive for:
improving a Li-ion battery life; and/or
improving a Li-ion battery cycling stability; and/or
reducing a Li-ion battery irreversible capacity;

especially in a temperature range greater than or equal to 25° C., preferably between 25° C. and 65° C., advantageously between 40° C. and 60° C.;

the composition being such that:
the lithium 4,5-dicyano-2-(trifluoromethyl)imidazolate and lithium hexafluorophosphate total concentration is less than or equal to 1 mol/L relative to the total volume of composition; and
the lithium 4,5-dicyano-2-(trifluoromethyl)imidazolate concentration is less than or equal to 0.3 mol/L, preferably less than or equal to 0.05 mol/L, relative to the total volume of composition.

### BRIEF DESCRIPTION OF THE FIGURES

FIG. 1 shows the variation in discharge capacity as a function of the number of cycles performed at 45° C. as described in Example 1.

FIG. 2 shows the variation in discharge capacity as a function of the number of cycles performed at 60° C. as described in Example 2.

FIG. 3 shows the variation in discharge capacity as a function of the number of cycles performed at 25° C. as described in Example 3.

FIG. 4 shows the variation in discharge capacity as a function of the number of cycles performed at 40° C. as described in Example 3.

FIG. 5 shows the variation in discharge capacity as a function of the number of cycles performed at 60° C. as described in Example 3.

### DETAILED DESCRIPTION

The present application describes electrolyte compositions comprising specific concentration and ratio of two lithium salts, a solvent (that may be a mixture of solvents) and an electrolytic additive. More specifically, the electrolyte composition comprises lithium hexafluorophosphate (LiPF₆), lithium 4,5-dicyano-2-(trifluoromethyl)imidazolate (LiTDI), at least one solvent, and at least one electrolytic additive. The electrolyte composition as described herein comprises:
a total concentration of lithium hexafluorophosphate and of lithium 4,5-dicyano-2-(trifluoromethyl)imidazolate of less than or equal to 1 mol/L relative to the total volume of composition (i.e., [LiPF₆]+[LiTDI]≤1 mol/L); and
a lithium 4,5-dicyano-2-(trifluoromethyl)imidazolate concentration of less than or equal to 0.3 mol/L relative to the total volume of composition (i.e., 0<[LiTDI]≤0.3 mol/L).

For example, the lithium 4,5-dicyano-2-(trifluoromethyl)imidazolate content is less than or equal to 0.25 mol/L, or less than or equal to 0.2 mol/L, particularly less than or equal to 0.15 mol/L, or less than or equal to 0.1 mol/L, preferably less than or equal to 0.08 mol/L, preferably less than or equal to 0.05 mol/L, relative to the total volume of composition.

The lithium hexafluorophosphate concentration in the electrolyte composition may be greater than or equal to 0.80 mol/L and less than 1 mol/L, preferably comprised between 0.80 and less than 1 mol/L, particularly between 0.90 and 0.99 mol/L, and for example comprised between 0.95 mol/L and 0.99 mol/L, relative to the total volume of composition.

Examples of lithium hexafluorophosphate and lithium 4,5-dicyano-2-(trifluoromethyl)imidazolate concentrations in the electrolyte composition comprise:
0.99 mol/L of LiPF₆ and 0.01 mol/L of LiTDI;
0.98 mol/L of LiPF₆ and 0.02 mol/L of LiTDI;
0.97 mol/L of LiPF₆ and 0.03 mol/L of LiTDI;
0.96 mol/L of LiPF₆ and 0.04 mol/L of LiTDI;
0.95 mol/L of LiPF₆ and 0.05 mol/L of LiTDI;
0.90 mol/L of LiPF₆ and 0.1 mol/L of LiTDI;
0.80 mol/L of LiPF₆ and 0.2 mol/L of LiTDI; and
0.7 mol/L of LiPF₆ and 0.3 mol/L of LiTDI.

According to a preferred embodiment, the electrolyte composition as described in the present application comprises 0.95 mol/L of LiPF₆ and 0.05 mol/L of LiTDI, relative to the total volume of composition.

According to one embodiment, the solvent is non aqueous (organic). For example, the composition solvent may be selected from the group consisting of ethers, carbonic acid esters, cyclic carbonate esters, aliphatic carboxylic acid esters, aromatic carboxylic acid esters, phosphoric acid esters, sulfite esters, nitriles, amides, alcohols, sulfoxides, sulfolane, nitromethane, 1,3-dimethyl-2-imidazolidinone, 1,3-dimethyl-3,4,5,6-tetrahydro-2(1,H)-pyrimidinone, 3-methyl-2-oxazolidinone, or one of their mixtures.

Among ethers, mention may be made of linear or cyclic ethers such as, for example, dimethoxyethane (DME), methyl ethers of oligoethylene glycols of 2 to 5 oxyethylene units, dioxolane, dioxane, dibutyl ether, tetrahydrofuran, and their mixtures.

Among nitriles, mention may be made, for example, of acetonitrile, pyruvonitrile, propionitrile, methoxypropionitrile, dimethylaminopropionitrile, butyronitrile, isobutyronitrile, valeronitrile, pivalonitrile, isovaleronitrile, glutaronitrile, methoxyglutaronitrile, 2-methylglutaronitrile, 3-methylglutaronitrile, adiponitrile, malononitrile, and their mixtures.

Examples of solvents also include those selected from the group consisting of dimethyl carbonate, ethyl methyl carbonate, diethyl carbonate, diphenyl carbonate, methyl phenyl carbonate, ethylene carbonate, propylene carbonate, butylene carbonate, vinylene carbonate, methyl formate, methyl acetate, methyl propionate, ethyl acetate, butyl acetate, and their mixtures. The solvent may also be selected from ethylene carbonate (EC-CAS N° 96-49-1), diethyl carbonate (DEC-CAS N° 105-58-8), and their mixtures. Preferably, the solvent is an ethylene carbonate:diethyl carbonate mixture in a ratio of between 1:99 and 99:1, preferably between 10:90 and 90:10, more preferably between 40:60 and 60:40.

Examples of electrolytic additive include fluoroethylene carbonate (FEC), vinylene carbonate, 4-vinyl-1,3-dioxolan-2-one, allyl ethyl carbonate, vinyl acetate, divinyl adipate, acrylonitrile, 2-vinylpyridine, maleic anhydride, methyl cinnamate, phosphonates, vinyl containing silane compounds, 2-cyanofurane and their mixtures, the electrolytic additive preferably being fluoroethylene carbonate (FEC). The electrolytic additive content may be comprised between 0.1% and 9%, preferably between 0.5% and 4%, by weight relative to the combined “solvent(s)+additive” total weight. Particularly, the electrolytic additive content in the electrolyte composition is less than or equal to 2% by weight relative to the combined “solvent(s)+additive” total weight.

According to an embodiment, the present electrolyte composition is selected from one of the following compositions (LiPF₆ and LiTDI concentrations being expressed relative to the total composition volume and the additive content relative to the combined “solvent(s)+additive” total weight):
i. 0.99 mol/L of LiPF₆ and 0.01 mol/L of LiTDI, FEC as electrolytic additive (particularly at a concentration less than or equal to 2% by weight), and an EC/DEC mixture as solvent;
ii. 0.98 mol/L of LiPF₆ and 0.02 mol/L of LiTDI, FEC as electrolytic additive (particularly at a concentration less than or equal to 2% by weight), and an EC/DEC mixture as solvent;
iii. 0.97 mol/L of LiPF₆ and 0.03 mol/L of LiTDI, FEC as electrolytic additive (particularly at a concentration less than or equal to 2% by weight), and an EC/DEC mixture as solvent;
iv. 0.96 mol/L of LiPF₆ and 0.04 mol/L of LiTDI, FEC as electrolytic additive (particularly at a concentration less than or equal to 2% by weight), and an EC/DEC mixture as solvent;
v. 0.95 mol/L of LiPF₆ and 0.05 mol/L of LiTDI, FEC as electrolytic additive (particularly at a concentration less than or equal to 2% by weight), and an EC/DEC mixture as solvent;
vi. 0.90 mol/L of LiPF₆ and 0.1 mol/L of LiTDI, FEC as electrolytic additive (particularly at a concentration less than or equal to 2% by weight), and an EC/DEC mixture as solvent;
vii. 0.80 mol/L of LiPF₆ and 0.2 mol/L of LiTDI, FEC as electrolytic additive (particularly at a concentration less than or equal to 2% by weight), and an EC/DEC mixture as solvent; and
viii. 0.7 mol/L of LiPF₆ and 0.3 mol/L of LiTDI, FEC as electrolytic additive (particularly at a concentration less than or equal to 2% by weight), and an

EC/DEC mixture as solvent.

The electrolyte composition may be prepared by dissolving, preferably with stirring, the salts in the appropriate proportions of solvent(s) comprising the electrolytic additive. Alternatively, the electrolyte composition may be prepared by dissolving, preferably with stirring, the salts and the electrolytic additive in appropriate proportions of solvent(s).

The use of an electrolyte composition of the present application in a Li-ion battery is also contemplated, in particular in a temperature range of higher than or equal to 25° C., preferably of between 25° C. and 65° C., advantageously between 40° C. and 60° C. For example, use is made in mobile devices, for instance mobile phones, cameras, tablets or laptops, in electric vehicles, or for the storage of renewable energy.

According to another aspect, the present application thus also relates to an electrochemical cell comprising a negative electrode, a positive electrode, and an electrolyte composition as defined herein, interposed between the negative electrode and the positive electrode. The electrochemical cell may also further comprise a separator in which the electrolyte composition of the present application is impregnated.

The present application also contemplates a battery comprising at least one electrochemical cell defined in this application. When the battery comprises several of these electrochemical cells, said cells can be assembled serially and/or in parallel.

In the context of the present application, by negative electrode is meant the electrode which acts as anode, when the battery delivers current (i.e. when in discharge process) and which acts as cathode when the battery is in charging process. The negative electrode typically comprises an electrochemically active material, optionally an electronically conductive material, and optionally a binder. The term “electrochemically active material” means a material capable of reversibly inserting ions, without irreversibly damaging their structure. By “electronically conductive material” is meant a material capable of conducting electrons.

For example, the battery negative electrode may comprise as electrochemically active material, graphite, carbon fibers, carbon black, or a mixture thereof, the negative electrode preferably comprising graphite. The negative electrode may also comprise lithium, which may then consist of a metallic lithium film or of an alloy comprising lithium. A negative electrode example comprises an active lithium film prepared by rolling a lithium foil between rolls.

In the context of the present application, by positive electrode is meant the electrode which acts as a cathode when the battery delivers current (i.e. when in discharge process) and which acts as the anode when the battery is in charging process. The positive electrode usually comprises an electrochemically active material, optionally an electronically conductive material, and optionally a binder.

The electrochemical cell's positive electrode may comprise an electrochemically active material selected from LiCoO₂, LiFePO₄ (LFP), LiMnₓCo_{y}Ni_{z}O₂ (NMC, with x+y+z=1), LiFePO₄F, LiFeSO₄F, LiNiCoAlO₂ and their mixtures.

In addition to the electrochemically active material, the positive electrode material may also comprise an electronically conductive material such as a carbon source, including, for example, carbon black, Ketjen® carbon, Shawinigan carbon, graphite, graphene, carbon nanotubes, carbon fibers (such as vapor grown carbon fibers (VGCF)), non-powdery carbon obtained by carbonizing an organic precursor, or a combination of at least two thereof. Other additives may also be present in the positive electrode material, such as lithium salts or inorganic particles of ceramic or glass type, or other compatible active materials (for example, sulfur).

The positive electrode material may also comprise a binder. Non-limiting examples of binders include linear, branched and/or crosslinked polyether polymer binders (e.g., polymers based on poly(ethylene oxide) (PEO), or poly(propylene oxide) (PPO) or a mixture of both (or a EO/PO copolymer), and optionally comprising crosslinkable units), water soluble binders (such as SBR (styrene-butadiene rubber), NBR (acrylonitrile-butadiene rubber), HNBR (hydrogenated NBR), CHR (epichlorohydrin rubber), ACM (acrylate rubber)), or fluorinated polymer type binders (such as PVDF (polyvinylidene fluoride), PTFE (polytetrafluoroethylene), and their combinations). Some binders, such as those soluble in water, may also include an additive such as CMC (carboxymethylcellulose).

According to one embodiment, the electrochemical cell comprises a negative electrode containing graphite, a positive electrode containing LiMnₓCo_{y}Ni_{z}O₂ (NMC, with x+y+z=1), and an electrolyte composition as herein defined, interposed between the negative electrode and positive electrode, the composition being preferably selected from any of the following compositions (LiPF₆ and LiTDI concentrations being expressed relative to the total volume of composition and the additive content expressed relative to the “solvent(s)+additive” total combined weight):
i. 0.99 mol/L of LiPF₆ and 0.01 mol/L of LiTDI, FEC as electrolytic additive (particularly at a concentration less than or equal to 2% by weight), and an EC/DEC mixture as solvent;
ii. 0.98 mol/L of LiPF₆ and 0.02 mol/L of LiTDI, FEC as electrolytic additive (particularly at a concentration less than or equal to 2% by weight), and an EC/DEC mixture as solvent;
iii. 0.97 mol/L of LiPF₆ and 0.03 mol/L of LiTDI, FEC as electrolytic additive (particularly at a concentration less than or equal to 2% by weight), and an EC/DEC mixture as solvent;
iv. 0.96 mol/L of LiPF₆ and 0.04 mol/L of LiTDI, FEC as electrolytic additive (particularly at a concentration less than or equal to 2% by weight), and an EC/DEC mixture as solvent;
v. 0.95 mol/L of LiPF₆ and 0.05 mol/L of LiTDI, FEC as electrolytic additive (particularly at a concentration less than or equal to 2% by weight), and an EC/DEC mixture as solvent;
vi. 0.90 mol/L of LiPF₆ and 0.1 mol/L of LiTDI, FEC as electrolytic additive (particularly at a concentration less than or equal to 2% by weight), and an EC/DEC mixture as solvent;
vii. 0.80 mol/L of LiPF₆ and 0.2 mol/L of LiTDI, FEC as electrolytic additive (particularly at a concentration less than or equal to 2% by weight), and an EC/DEC mixture as solvent; and
viii. 0.7 mol/L of LiPF₆ and 0.3 mol/L of LiTDI, FEC as electrolytic additive (particularly at a concentration less than or equal to 2% by weight), and an EC/DEC mixture as solvent.

According to another embodiment, the electrochemical cell comprises a negative electrode containing graphite, a positive electrode containing LiFePO₄ (LFP) and a mixture of carbon black with carbon fibers and/or carbon nanotubes, and an electrolyte composition as defined herein, interposed between the negative electrode and positive electrode, the composition preferably being selected from any of the following compositions (LiPF₆ and LiTDI concentrations being expressed relative to the total volume of composition and the additive content expressed relative to the “solvent(s)+additive” total combined weight):
i. 0.99 mol/L of LiPF₆ and 0.01 mol/L of LiTDI, FEC as electrolytic additive (particularly at a concentration less than or equal to 2% by weight), and an EC/DEC mixture as solvent;
ii. 0.98 mol/L of LiPF₆ and 0.02 mol/L of LiTDI, FEC as electrolytic additive (particularly at a concentration less than or equal to 2% by weight), and an EC/DEC mixture as solvent;
iii. 0.97 mol/L of LiPF₆ and 0.03 mol/L of LiTDI, FEC as electrolytic additive (particularly at a concentration less than or equal to 2% by weight), and an EC/DEC mixture as solvent;
iv. 0.96 mol/L of LiPF₆ and 0.04 mol/L of LiTDI, FEC as electrolytic additive (particularly at a concentration less than or equal to 2% by weight), and an EC/DEC mixture as solvent;
v. 0.95 mol/L of LiPF₆ and 0.05 mol/L of LiTDI, FEC as electrolytic additive (particularly at a concentration less than or equal to 2% by weight), and an EC/DEC mixture as solvent;
vi. 0.90 mol/L of LiPF₆ and 0.1 mol/L of LiTDI, FEC as electrolytic additive (particularly at a concentration less than or equal to 2% by weight), and an EC/DEC mixture as solvent;
vii. 0.80 mol/L of LiPF₆ and 0.2 mol/L of LiTDI, FEC as electrolytic additive (particularly at a concentration less than or equal to 2% by weight), and an EC/DEC mixture as solvent; and
viii. 0.7 mol/L of LiPF₆ and 0.3 mol/L of LiTDI, FEC as electrolytic additive (particularly at a concentration less than or equal to 2% by weight), and an EC/DEC mixture as solvent.

For example, the electrochemical cell as described herein may have a capacity retention greater than or equal to 80% after at least 500 charge/discharge cycles compared to the first cycle, for a charge comprised between a voltage V_{low} between 2.0 and 3.0 volts versus Li⁺/Li⁰, and a voltage V_{high} between 3.8 and 4.2 volts versus Li⁺/Li⁰, at a temperature of 45° C., and at a charge and discharge C rate. In particular, the voltage V_{low} may be of 2.8 volts and the voltage V_{high} is of 4.2 volts, the positive electrode preferably comprising LiCoO₂, LiMnₓCo_{y}Ni_{z}O₂ (with x+y+z=1), LiFePO₄F, LiFeSO₄F, LiNiCoAlO₂ or their mixtures.

According to another embodiment, the electrochemical cell as described herein may have a capacity retention greater than or equal to 80% after at least 60 charge/discharge cycles compared to the first cycle, for a charge comprised between a voltage V_{low} between 2.0 and 3.0 volts versus Li⁺/Li⁰, and a voltage V_{high} between 3.8 and 4.2 volts versus Li⁺/Li⁰, at a temperature of 60° C., and at a charge and discharge C/4 rate, charging being optionally followed by the application of a constant voltage of 4.2V for 1 h. In particular, the voltage V_{low} is of 2.8 volts and the voltage V_{high} is of 4.2 volts, the positive electrode preferably being selected from the group consisting of LiCoO₂, LiMnₓCo_{y}Ni_{z}O₂ (with x+y+z=1), LiFePO₄F, LiFeSO₄F, LiNiCoAlO₂ and mixtures thereof. According to one example, charging is followed by the application of a constant voltage as described.

In another example, the electrochemical cell of the present technology has a capacity retention greater than or equal to 80% after at least 500 charge/discharge cycles compared to the first cycle, for a charge comprised between a voltage V_{low} of between 2.0 and 3.0 volts versus Li⁺/Li⁰, and a voltage V_{high} of between 3.8 and 4.2 volts versus Li⁺/Li⁰, at a temperature of 25° C., and at a charge and discharge rate of C, charging being optionally followed by the application of a constant voltage of 4V during 30 minutes, the positive electrode preferably comprising LiFePO₄. Particularly, the voltage V_{low} may be equal to 2 volts and the voltage V_{high} is of 4 volts. According to an example, charging is followed by the application of a constant voltage as described.

The electrochemical cell of the present technology may also have a capacity retention greater than or equal to 80% after at least 200 charge/discharge cycles relative to the first cycle, for a charge comprised between a voltage V_{low} between 2.0 and 3.0 volts versus Li⁺/Li⁰, and a voltage V_{high} between 3.8 and 4.2 volts versus Li⁺/Li⁰, at a temperature of 40° C., and at a charge and discharge C rate, charging being optionally followed by the application of a constant voltage of 4V during 30 minutes, the positive electrode preferably comprising LiFePO₄. Particularly, the voltage V_{low} is equal to 2 volts and the voltage V_{high} is of 4 volts. According to an example, charging is followed by the application of a constant voltage as described.

The electrochemical cell of the present technology may have a capacity retention greater than or equal to 80% after at least 100 charge/discharge cycles relative to the first cycle, for a charge comprised between a voltage V_{low} between 2.0 and 3.0 volts versus Li⁺/Li⁰, and a voltage V_{high} between 3.8 and 4.2 volts versus Li⁺/Li⁰, at a temperature of 60° C., and at a charge and discharge C rate, charging being optionally followed by the application of a constant voltage of 4V during 30 minutes, the positive electrode preferably comprising LiFePO₄. Particularly, the voltage V_{low} is equal to 2 volts and the voltage V_{high} is of 4 volts. According to an example, charging is followed by the application of a constant voltage as described.

The present application also relates to the use of the electrolyte composition as described herein for:
improving a Li-ion battery life; and/or
improving a Li-ion battery cycling stability; and/or
reducing a Li-ion battery irreversible capacity;

especially in a temperature range above or equal to 25° C., preferably between 25° C. and 65° C., advantageously between 40° C. and 60° C.

Another aspect relates to the use of lithium 4,5-dicyano-2-(trifluoromethyl)imidazolate in an electrolyte composition comprising lithium hexafluorophosphate and at least one electrolytic additive for:
improving a Li-ion battery life; and/or
improving a Li-ion battery cycling stability; and/or
reducing a Li-ion battery irreversible capacity;

especially in a temperature range above or equal to 25° C., preferably comprised between 25° C. and 65° C., advantageously between 40° C. and 60° C.;

the composition being such that:
the lithium 4,5-dicyano-2-(trifluoromethyl)imidazolate and lithium hexafluorophosphate total concentration is less than or equal to 1 mol/L; and
the lithium 4,5-dicyano-2-(trifluoromethyl)imidazolate concentration is less than or equal to 0.3 mol/L, preferably less than or equal to 0.05 mol/L.

According to an example, the use of lithium 4,5-dicyano-2-(trifluoromethyl)imidazolate in an electrolyte composition as herein described and comprising lithium hexafluorophosphate and at least one electrolytic additive, makes it possible to improve the life duration of a Li-ion battery; and/or to improve the stability to cycling of a Li-ion battery; and/or to reduce the irreversible capacity of a Li-ion battery. This improvement may occur especially in a temperature range above or equal to 25° C., preferably comprised between 25° C. and 65° C., advantageously between 40° C. and 60° C. For instance, the presence of LiTDI in the electrolyte composition makes it possible to increase the life of the battery (80% loss of initial capacity) by at least 1.5-fold, or at least 2-fold, compared to a battery without LiTDI used in the same conditions. According to another example, the battery life is multiplied by at least 1.5, or at least 2, or multiplied by a number within the range of from 1.5 to 8, or from 2 to 7.

It is understood that measurable or quantifiable values, such as concentrations, volumes, etc. mentioned in this application must be interpreted taking into account the limitations of the analysis method and the uncertainty inherent to the instrument used.

All embodiments and alternatives described above can be combined with each other. In particular, the various embodiments and alternatives of the various composition elements can be combined with each other, as well as for the use of said composition.

For the purposes of this document, “between x and y”, or “from x to y”, means an interval in which the x and y terminals are included. For example, the range “between 1 and 4%” namely includes the values 1 and 4%.

The following examples are for illustrative purposes and should not be interpreted as limiting the scope of the invention as described.

### EXAMPLES

### Example 1

The first example carried out consists in dissolving, at room temperature, a salt mixture containing LiPF₆ and LiTDI (or LiPF₆ alone as a reference) at a total concentration of 1 mol/L, in a mixture of three carbonates: ethylene carbonate (EC), diethyl carbonate (DEC) and fluoroethylene carbonate (FEC) in EC/DEC/FEC weight proportions of: 36.84%, 61.16% and 2% respectively.

Four mixtures were thus prepared in this example in the following proportions:
1 mol/L of LiPF₆
0.95 mol/L of LiPF₆ and 0.05 mol/L of LiTDI
0.9 mol/L of LiPF₆ and 0.1 mol/L of LiTDI
0.8 mol/L of LiPF₆ and 0.2 mol/L of LiTDI

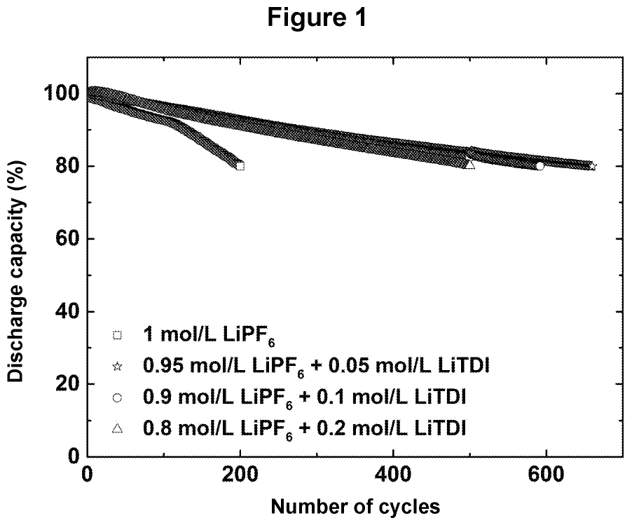

These mixtures were electrochemically evaluated in lithium-ion pouch-cells of 11.5mAh capacity, with NMC and graphite as cathode and anode materials respectively. The system's cycling terminals are of 2.8-4.2V. After a slow rate (C/24) formation at room temperature, the mixtures were evaluated at 45° C. with a C charge and discharge. Results obtained are presented in FIG. 1. If the end of life of a battery is considered to be when it has lost 80% of its initial capacity, the addition of LiTDI can multiply by 2.5 to 3.3 the life of the battery. The use of LiTDI at a content of only 0.05 mol/L makes it possible to carry out more than 600 cycles at the battery end of life.

### Example 2

The second example carried out consists in dissolving at room temperature a salt mixture containing LiPF₆ and LiTDI (or LiPF₆ alone for the reference) at a total concentration of 1 mol/L, in a mixture of three carbonates: ethylene carbonate (EC), diethyl carbonate (DEC) and fluoroethylene carbonate (FEC) in weight proportions of 36.84%, 61.16% and 2% respectively.

The following four mixtures were prepared:
1 mol/L of LiPF₆
0.95 mol/L of LiPF₆ and 0.05% mol/L of LiTDI
0.9 mol/L of LiPF₆ and 0.1 mol/L of LiTDI
0.7 mol/L of LiPF₆ and 0.3 mol/L of LiTDI

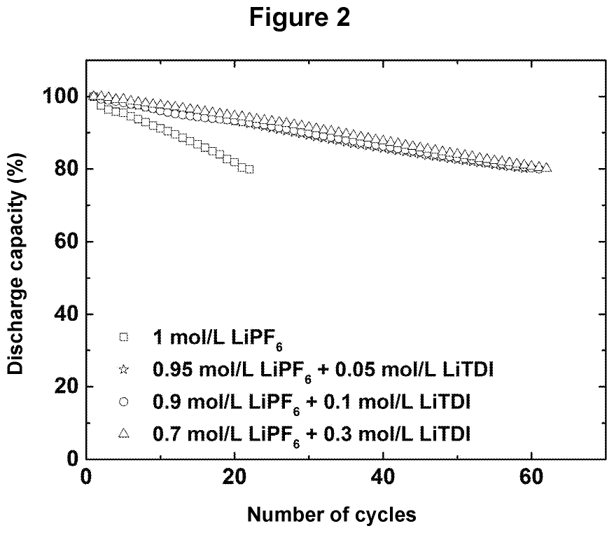

These mixtures were electrochemically evaluated in lithium-ion pouch-cells of 11.5 mAh capacity, with NMC and graphite as cathode and anode materials respectively. The system's cycling terminals are 2.8-4.2V. After a slow rate (C/24) formation at room temperature, the mixtures were evaluated at 60° C. with a C/4 charge followed by application of a constant voltage at 4.2 V for 1 hour, and then a C/4 discharge. FIG. 2 shows the results obtained. If the end of life of a battery is considered to be when it has lost 80% of its initial capacity, the addition of LiTDI can multiply by 3 the life of the battery.

### Example 3

A salt mixture containing LiPF₆ and LiTDI (or LiPF₆ alone for the reference) is dissolved at a total concentration of 1 mol/L in a mixture of three carbonates: ethylene carbonate (EC), diethyl carbonate (DEC) and fluoroethylene carbonate (FEC) in weight proportions of 36.84%, 61.16% and 2% respectively.

Three mixtures were prepared in this example in the following proportions:
1 mol/L of LiPF₆
0.95 mol/L of LiPF₆ and 0.05 mol/L of LiTDI
0.8 mol/L of LiPF₆ and 0.2 mol/L of LiTDI

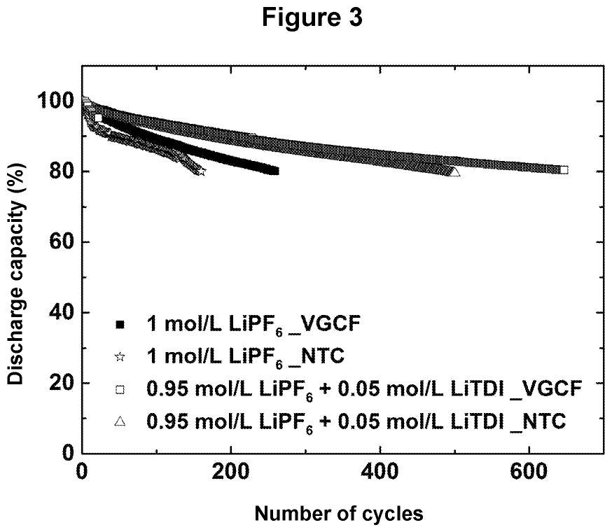

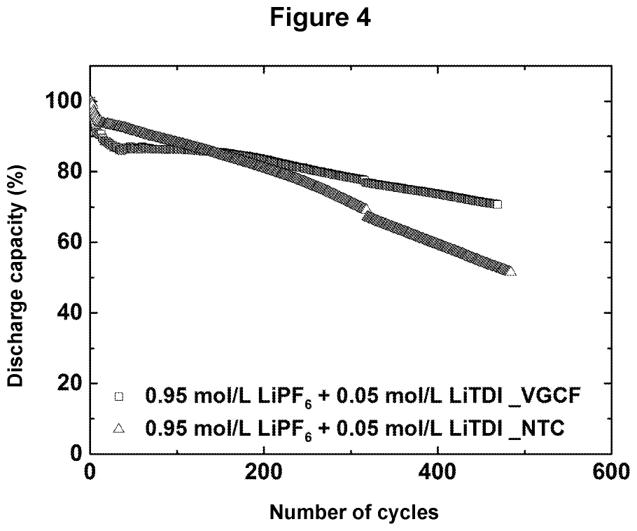

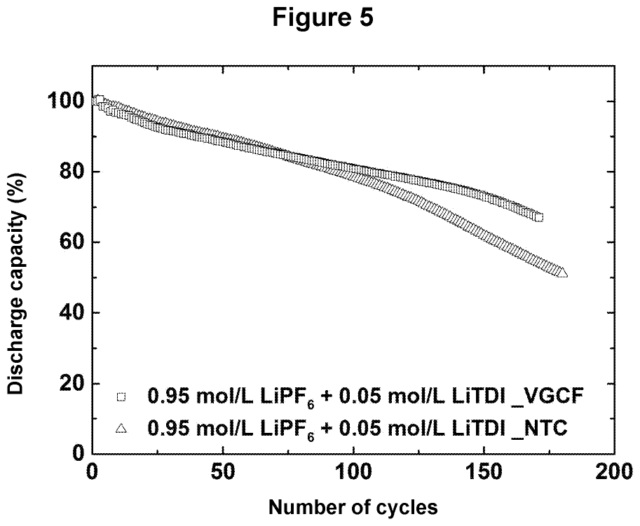

These mixtures were electrochemically evaluated in lithium-ion pouch-cells of 10 mAh capacity, with LFP and graphite as cathode and anode materials respectively. For the cathode, the electronic conductor used is a mixture of carbon black with either carbon fibers or nanotubes. The system's cycling terminals are 2-4V. After a slow rate (C/24) formation at room temperature, the mixtures were evaluated at 25, 40 and 60° C. with a C charge followed by application of a constant voltage at 4 V for 30 minutes, and then a C discharge. Results obtained are presented in FIGS. 3, 4 and 5 respectively (results shown for cells comprising 0.05 mol/L of LiTDI).

If the end of life of a battery is considered to be when it has lost 80% of its initial capacity, at 25° C. the addition of LiTDI at only 0.05 mol/L makes it possible to multiply by 3.2 the life of the battery with carbon nanotubes as electronic conductor and by 2.5 with the carbon fibers. The improvement in cyclability is more pronounced in the presence of carbon nanotubes where the battery life is increased by 4.2 times by adding 0.2 mol/L of LiTDI. At 40 and 60° C., the addition of 0.05 mol/L of LiTDI is sufficient to improve the cycle life of a few tens of cycles, whether with VGCF or CNT electronic conductors.

In summary, the effect of the LiTDI lithium salt on battery life has been demonstrated in the various series of electrochemical tests carried out on 10 mAh or 11.5 mAh capacity pouch-cells. The systems studied are LFP (with carbon black and CNT or VGCF)/graphite and NMC/graphite. Tests were carried out between 25° C. and 60° C., with or without applying a constant voltage after charging.

It has been shown that the addition of LiTDI (from 0.05 mol/L) makes it possible to significantly improve battery life. Without wishing to be bound by theory, it seems that the presence of LiTDI could allow to capture water molecules and prevent HF formation that occurs when LiPF₆ reacts with traces of moisture which may be contained in the cathodes, anodes, separator, solvent, packaging, etc. Unlike LiPF₆, LiTDI does not seem to be affected by the presence of moisture and can increase the life of the battery even at low concentration.

The series of tests carried out also demonstrates the good resistance in abusive cycling (application of constant voltage at the end of charging) of the tested electrolytes when containing LiTDI (from 0.05 mol/L). The tests carried out at room temperature on the LFP/graphite system further demonstrate the resistance to abusive cycling (no temperature effect) of electrolytes containing LiTDI, whether with VGCF or CNT type electronic conductors; the life of the battery being multiplied by 2.5 or 3.2 times.

Several modifications could be made to any of the above described embodiments without departing from the scope of the present invention as contemplated. Any references, patents or scientific literature documents referred to in the present application are incorporated herein by reference in their entirety and for all purposes.

1. Electrolyte composition comprising lithium hexafluorophosphate, lithium 4,5-dicyano-2-(trifluoromethyl)imidazolate, at least one solvent, and at least one electrolytic additive, said composition comprising:
a total concentration of lithium hexafluorophosphate and lithium 4,5-dicyano-2-(trifluoromethyl)imidazolate less than or equal to 1 mol/L relative to the total volume of the composition, and
a lithium 4,5-dicyano-2-(trifluoromethyl)imidazolate concentration less than or equal to 0.3 mol/L relative to the total volume of the composition.

2. Composition according to claim 1, wherein the content of lithium 4,5-dicyano-2-(trifluoromethyl)imidazolate is less than or equal to 0.2 mol/L, in particular less than or equal to 0.1 mol/L, preferably less than or equal to 0.08 mol/L, more preferably less than or equal to 0.05 mol/L, relative to the total volume of the composition.

3. Composition according to claim 1, wherein the solvent is selected from the group consisting of ethers, carbonic acid esters, cyclic carbonate esters, aliphatic carboxylic acid esters, aromatic carboxylic acid esters, phosphoric acid esters, sulfite esters, nitriles, amides, alcohols, sulfoxides, sulfolane, nitromethane, 1,3-dimethyl-2-imidazolidinone, 1,3-dimethyl-3,4,5,6-tetrahydro-2(1,H)-pyrimidinone, 3-methyl-2-oxazolidinone, and mixtures thereof.

4. Composition according to claim 3, wherein the solvent is selected from the group consisting of dimethyl carbonate, ethyl methyl carbonate, diethyl carbonate, diphenyl carbonate, methyl phenyl carbonate, ethylene carbonate, propylene carbonate, butylene carbonate, vinylene carbonate, methyl formate, methyl acetate, methyl propionate, ethyl acetate, butyl acetate, and mixtures thereof.

5. Composition according to claim 4, wherein the solvent is selected from the group consisting of ethylene carbonate, diethyl carbonate, and mixtures thereof.

6. Composition according to claim 1, wherein the electrolytic additive is selected from the group consisting of fluoroethylene carbonate, vinylene carbonate, 4-vinyl-1,3-dioxolan-2-one, allyl ethyl carbonate, vinyl acetate, divinyl adipate, acrylonitrile, 2-vinylpyridine, maleic anhydride, methyl cinnamate, phosphonates, vinyl containing silane compounds, 2-cyanofurane and mixtures thereof, the electrolytic additive preferably being fluoroethylene carbonate.

7. Composition according to claim 1 wherein the content of electrolytic additive is comprised between 0.1% and 9%, preferably between 0.5% and 4% by weight relative to the “solvent(s)+additive” total combined weight.

8. Composition according to claim 1, wherein the concentration of lithium hexafluorophosphate is greater than or equal to 0.80 mol/L and less than 1 mol/L, preferably comprised between 0.80 and less than 1 mol/L, particularly between 0.90 and 0.99 mol/L, and for example comprised between 0.95 mol/L and 0.99 mol/L, relative to the total volume of the composition.

9. Composition according to claim 1, wherein the lithium hexafluorophosphate concentration is of 0.95 mol/L, and the lithium 4,5-dicyano-2-(trifluoromethyl)imidazolate concentration is of 0.05 mol/L, relative to the total volume of the composition.

10. Use of a composition according to claim 1, in a Li-ion battery, particularly in a temperature range above or equal to 25° C., preferably comprised between 25° C. and 65° C., advantageously between 40° C. and 60° C.

11. Use according to claim 10, in mobile devices, for instance mobile phones, cameras, tablets or laptops, in electric vehicles, or in the storage of renewable energy.

12. Use of a composition according to claim 1, for:
improving a Li-ion battery life; and/or
improving a Li-ion battery cycling stability; and/or
reducing a Li-ion battery irreversible capacity;
particularly in a temperature range above or equal to 25° C., preferably comprised between 25° C. and 65° C., advantageously between 40° C. and 60° C.

13. Electrochemical cell comprising a negative electrode, a positive electrode, and an electrolyte composition as defined in claim 1, interposed between the negative electrode and the positive electrode.

14. Electrochemical cell according to claim 13, wherein the negative electrode comprises graphite, carbon fibers, carbon black, lithium, or a mixture thereof, the negative electrode preferably comprising graphite.

15. Electrochemical cell according to claim 13, wherein the positive electrode comprises LiCoO₂, LiFePO₄, LiMnₓCo_{y}Ni_{z}O₂ (where x+y+z=1), LiFePO₄F, LiFeSO₄F, LiNiCoAlO₂ or a mixture thereof, the positive electrode preferably comprising LiFePO₄ or LiMnₓCo_{y}Ni_{z}O₂ (where x+y+z=1).

16. Electrochemical cell according to claim 13, having a capacity retention greater than or equal to 80% after at least 500 charge/discharge cycles compared to the first cycle, for a charge comprised between a voltage V_{low} between 2.0 and 3.0 volts versus Li⁺/Li⁰, and a voltage V_{high} between 3.8 and 4.2 volts versus Li⁺/Li⁰, at a temperature of 25° C., and at a charge and discharge rate of C.

17. Electrochemical cell according to claim 16, wherein the voltage V_{low} is equal to 2.8 volts and the voltage V_{high} is equal to 4.2 volts, the positive electrode preferably comprising LiCoO₂, LiMnₓCo_{y}Ni_{z}O₂(with x+y+z=1), LiFePO₄F, LiFeSO₄F, LiNiCoAlO₂ or their mixtures.

18. Electrochemical cell according to claim 16, having a capacity retention greater than or equal to 80% after at least 500 charge/discharge cycles compared to the first cycle, for a charge comprised between a voltage V_{low} between 2.0 and 3.0 volts versus Li⁺/Li⁰, and a voltage V_{high} between 3.8 and 4.2 volts versus Li⁺/Li⁰, at a temperature of 25° C., and at a charge and discharge rate of C, charging being optionally followed by the application of a constant voltage of 4V during 30 minutes, the positive electrode preferably comprising LiFePO₄.

19. Electrochemical cell according to claim 18, wherein the voltage V_{low} is equal to 2 volts and the voltage V_{high} is equal to 4 volts.

20. Electrochemical cell according to claim 18, charging being followed by the application of a constant voltage of 4V for 30 minutes.

21. Electrochemical cell according to claim 18, charging not being followed by the application of a constant voltage of 4V for 30 minutes and the capacity retention being greater than or equal to 80% after at least 800 cycles.

22. Battery comprising at least one electrochemical cell according to claim 13.

23. Use of lithium 4,5-dicyano-2-(trifluoromethyl)imidazolate in an electrolyte composition comprising lithium hexafluorophosphate and at least one electrolytic additive for:
improving a Li-ion battery life; and/or
improving a Li-ion battery cycling stability; and/or
reducing a Li-ion battery irreversible capacity;
especially in a temperature range above or equal to 25° C., preferably between 25° C. and 65° C., advantageously between 40° C. and 60° C.;
the composition being such that:
the total concentration of lithium 4,5-dicyano-2-(trifluoromethyl)imidazolate and lithium hexafluorophosphate is less than or equal to 1 mol/L relative to the total volume of the composition; and
the lithium 4,5-dicyano-2-(trifluoromethyl)imidazolate concentration is less than or equal to 0.3 mol/L, preferably less than or equal to 0.05 mol/L, relative to the total volume of the composition.
